(19) United States
(12) Patent Application Publication
(10) Pub. No.: US 2011/0071432 A1
(43) Pub. Date: Mar. 24, 2011

(54) Fine Needle Aspiration Device with Distal Anchor

(76) Inventors: Oscar R. Carrillo, JR., Attleboro, MA (US); Adam L. Cohen, Arlington, MA (US); Michal Weisman, Palo Alto, CA (US); Yem Chin, Burlington, MA (US); Robert Devries, Northboro, MA (US)

(21) Appl. No.: 12/888,547

(22) Filed: Sep. 23, 2010

Related U.S. Application Data
(60) Provisional application No. 61/245,446, filed on Sep. 24, 2009.

(51) Int. Cl. A61B 10/02

(52) U.S. Cl. 600/567

(57) ABSTRACT

A device for capturing a tissue sample from within a body comprises a needle slidably comprising a needle lumen extending therethrough to a needle opening in the distal end and a stylet slidably received in the needle lumen for movement between an extended position in which a tissue penetrating distal tip of the stylet extends out of the needle opening to penetrate target tissue and a retracted position in which the distal tip of the stylet is received within the needle opening to substantially seal the needle lumen. The stylet further comprises an anchoring feature located proximally of the tissue penetrating distal tip. The anchoring feature comprises a first gripping member including a first proximal facing abutting surface adjacent to a first tissue receiving gap. Movement of the stylet distally out of the needle lumen brings the first gripping member into engagement with surrounding tissue to anchor the stylet at a desired position within the body.

### PRIORITY CLAIM

The application claims the priority to the U.S. Provisional Application Ser. No. 61/245,446, entitled FINE NEEDLE ASPIRATION DEVICE WITH DISTAL ANCHOR” filed Sep. 24, 2009. The specification of the above-identified application is incorporated herewith by reference.

### BACKGROUND

Catheters are often employed to inject fluids into and/or to obtain fluid and/or tissue samples from living bodies. In these procedures, a needle is advanced, for example, through a catheter to a target site under guidance from an endoscopic vision system or other imaging technique (e.g., ultrasound). The needle is then advanced distally from the catheter to penetrate the target site. In biopsy procedures, suction may then be applied (e.g., via a syringe) to draw sample tissue into the needle. Furthermore, the needle may be aspirated multiple times to enable the capture of tissue therein. During aspiration, it is vital that a position of the needle remain substantially constant to prevent the inadvertent sampling of non-targeted tissue and to minimize patient trauma.

### SUMMARY OF THE INVENTION

A device for capturing a tissue sample from within a body according to the present invention comprises a needle slidably comprising a needle lumen extending therethrough to a needle opening in the distal end. A stylet may be slidably received in the needle lumen for movement between an extended position in which a tissue penetrating distal tip of the stylet extends out of the needle opening to penetrate target tissue and a retracted position in which the distal tip of the stylet is received within the needle opening to substantially seal the needle lumen. The stylet may further comprise an anchoring feature located proximally of the tissue penetrating distal tip, the anchoring feature comprising a first gripping member including a first proximal facing abutting surface adjacent to a first tissue receiving gap wherein movement of the stylet distally out of the needle lumen brings the first gripping member into engagement with surrounding tissue to anchor the stylet at a desired position within the body.

### BRIEF DESCRIPTION OF THE DRAWINGS

FIG. 1 shows a side view of a device according to a first exemplary embodiment of the invention in a compressed configuration;

FIG. 2 shows a partial cut-through enlarged view of a distal tip of the device of FIG. 1 in a closed configuration;

FIG. 3 shows an enlarged view of a distal tip of the device of FIG. 1 in an open configuration;

FIG. 4 shows an enlarged view of a distal tip of a second exemplary embodiment of the present invention;

FIG. 5 shows an enlarged view of a distal tip of a third exemplary embodiment of the present invention;

FIG. 6 shows a side view of a the device of FIG. 5; and

FIG. 7 shows an enlarged view of a distal tip of a fourth exemplary embodiment of the present invention.

### DETAILED DESCRIPTION

The present invention, which may be further understood with reference to the following description and the appended drawings, relates to a device for the deployment and anchoring of a needle within a living body to perform a procedure such as, for example, a fine-needle biopsy. It is noted, however, that although the exemplary embodiments of the present invention are described with respect to particular procedures, the invention is not limited to these procedures and may be employed in any of a number of procedures requiring the insertion of a needle to a target site within the body. For example, embodiments of the present invention may be employed to remove target tissue from a body for treatment (e.g., removing a tumor) or for analysis.

Devices and methods according to exemplary embodiments of the invention teach anchoring a needle at a target site in a body via a stylet inserted therethrough and extending distally therefrom. A user of the device of the present invention operates an external control to push an anchoring stylet distally out of a needle once a target sampling site has been reached. The stylet may include one or more abutments or other shapes that anchor the stylet in place, as will be described in greater detail below. Anchoring the stylet at the target sampling site facilitates sampling target tissue by allowing proximal and distal movement of the needle while ensuring that a base position thereof remains substantially constant. Furthermore, the exemplary embodiment of the present invention also allows the stylet to aid in sampling target tissue by scraping tissue into grooves formed therein, as will also be described in greater detail below.

As shown in FIG. 1, a device 100 according to a first exemplary embodiment of the present invention comprises an elongated catheter shaft 106 with a lumen 110 extending longitudinally therethrough, the lumen 110 further housing a needle 102 therein. The lumen 110 of the shaft 106 extends from a proximal end (not shown) of the shaft 106 near a handle 130 of the device 100 to an opening at a distal end 104. It is noted that the use of the term distal herein refers to a direction away from a user of the device while the term proximal refers to a direction approaching a user of the device. The proximal portion of the device 100, including the handle 130, remains external to the body and accessible to the user while the distal portion, when in an operative position, extends into the body to a target site to be accessed (e.g., a site from which tissue samples are to be obtained in a biopsy procedure). The handle 130 is also provided with a gripping and manipulation member (not shown) as known in the art.

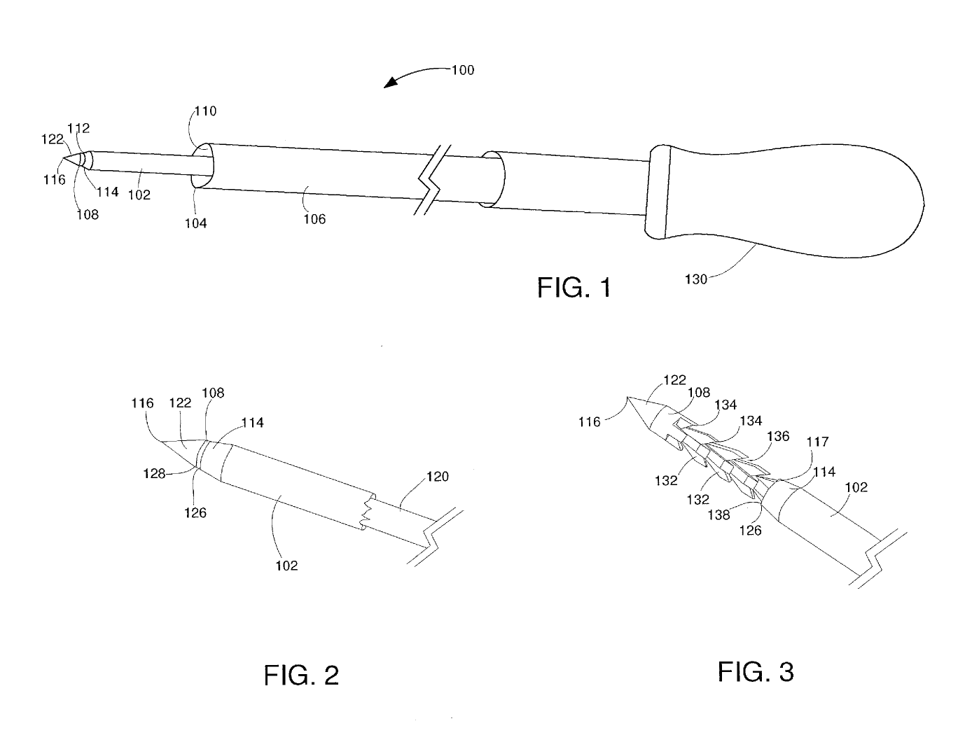

The needle 102 extends from a proximal end (not shown) in the handle 130 to a tapered distal end 114 surrounding a distal opening 117 of a needle lumen 112 extending therethrough to a proximal end (not shown) connectable to one or both of a receiving chamber and a source of suction, as those skilled in the art will understand. The needle lumen 112 and the distal opening 117 are dimensioned so that a stylet 108 as described below may be selectively inserted therethrough to form a fluid seal preventing entry into the needle lumen 112 of non-targeted tissue as the device 100 is moved to and from the target sampling site. The stylet 108 can be formed as an elongated tubular structure 120 extending from a proximal end (not shown) manipulable by a user to a distal end comprising a tapered distal portion 122 with a tissue penetrating tip 116 at a distal end thereof. The tapered distal portion 122 may, for example, be formed so that, when received in the distal opening 117 of the needle lumen 112, the tapered distal portion 122 and the tapered distal end 114 of the needle form a substantially continuous surface. Thus, when received in the distal opening 117 in a closed configuration as shown in FIG. 2, the distal tip 116 of the stylet 108 facilitates insertion of the needle 102 through tissue to reach the target site.

The needle 102 is movable between the closed configuration shown in FIG. 2 and an open configuration shown in FIG. 3 in which the stylet 108 is extended into tissue adjacent to the distal end 114, the device 100 is anchored to the this tissue and a base position of the needle 102 relative to the target sampling site is established. In the closed configuration, a distal-most rim 126 of the needle 102 surrounding the distal opening 117 of the needle lumen is adjacent to a proximal-most rim 128 of the tapered portion 122, wherein a diameter of the distalmost rim 126 is substantially equivalent to a diameter of the proximalmost rim 128 of the tapered tip 122 in order to create a substantially fluid-tight engagement therewith.

Once the target sampling site has been reached, the device 100 is moved to the open configuration shown in FIG. 3. Specifically, a position of the needle 102 may be held constant while the stylet 108 is moved distally relative thereto to penetrate into the tissue of the target sampling site (e.g., a tumor). Alternatively, the position of the stylet 108 may be held constant while the needle 102 is retracted proximally to expose the hooks 132 to the surrounding tissue. The stylet 108 and needle 102 may be relative to one another by a distance selected to embed in this tissue one or more of a plurality of hooks 132 formed along a distal portion of the stylet 108. The hooks 132 may be formed as a series of arms extending radially outward from an axis of the stylet 108 and angled proximally to act as barbs resisting movement of the stylet 108 relative to the tissue within which it is embedded as would be understood by those skilled in the art. The hooks 132 may be formed so that radially outer edges thereof will be no further from the axis of the stylet 108 than the proximal-most rim 128 of the tapered portion 122 so that the profile of the stylet 108 is not increased thereby and remains movable within the lumen 112. The hooks 132 may be distributed circumferentially around the stylet 108 and, in an exemplary embodiment, are provided in a plurality of sets of four hooks 132 evenly disposed circumferentially about the stylet 108 with the sets of four hooks 132 separated from one another longitudinally along the stylet 108 from a distal-most set adjacent to the proximal-most rim 128 along any desired length of the stylet 108. It is noted that each set of hooks 132 may include only one or any number of hooks and that the number of longitudinally separated sets of hooks 132 may be selected to obtain a desired anchoring force within the tissue to be sampled without deviating from the spirit and scope of the present invention. Pointed proximally facing tips 134 of the hooks 132 are sized and shaped to permit each of the hooks 132 to abut into the target tissue sampling site and anchor in place by providing a barb-like interface therewith. Furthermore, adjacent sets of hooks 132 may be separated from one another longitudinally by a gap 136 sized to permit the entry thereinto and gripping of tissue. The gaps 136 are also useful in retaining sampled tissue, as will be described in greater detail in a method taught hereinafter.

In accordance with an exemplary method of use of the device of FIGS. 1-3, the catheter shaft 106 is inserted into a living body and traversed to location adjacent to a target site at which tissue is, for example, to be sampled. During or after this insertion, the needle 102 is inserted through the lumen 110 of the catheter shaft 106 to a distal-most position therein with the stylet 108 in the closed configuration as shown in FIG. 2. The stylet 108 and the needle 102 may then be pushed further distally so that the puncturing tip 116 of the stylet 108 pierces the target tissue easing the insertion of the distal end 114 of the needle 102 thereinto. Once the needle 102 and the stylet 108 have been moved into the target tissue, the stylet 108 and the needle 102 are moved relative to one another to expose the hooks 132 and engage them with the target tissue by, for example, actuation of a control lever (not shown) on the handle 130. Specifically, actuation of the control lever (not shown) causes the stylet 108 to move relative to the needle 102, thus exposing the hooks 132 to the target tissue. Alternatively, the needle 102 may be moved by any other means known in the art. In a preferred embodiment, the needle 102 is retracted so that the distal end 114 thereof lies substantially adjacent a proximal end 138 of the hooks 132.

The user then moves the needle 102 distally and proximally over the stylet 108 to capture sample tissue within the needle lumen 112 while engagement between the surrounding tissue and the hooks 132 each time the hooks are exposed anchors the stylet 108 at a desired position within the target tissue. In this manner, the stylet 108 may be secured in place and the needle 102 may be moved back and forth multiple times to perform a biopsy and effectively dislodge a tissue sample from the surrounding tissue. Suction may be applied via any known apparatus to a proximal end of the needle lumen 112 to aspirate the captured samples from the proximal end for analysis or at least to move the captured samples to a portion of the needle lumen 112 within which they will be free from potentially damaging contact with the hooks 132 of the stylet 108. That is, the captured samples may be aspirated to an area of the needle lumen 112 which is located proximally of the proximal end 138 of the hooks 132 when the stylet 108 is retracted into the closed configuration. When the sampling has been completed, the stylet may be drawn proximally out of engagement with the surrounding tissue by applying thereto a proximally directed force (e.g., which holding the position of the needle 102 constant) sufficient to separate tissue engaged by the hooks 132 from surrounding tissue. Additional samples may then be obtained from the gaps 136 between adjacent sets of the hooks 132. Once the stylet 108 has been returned to the closed configuration with the hooks 132 positioned within the needle lumen 112, a further suction force may be applied to the needle lumen 112 to draw sampled tissue further proximally thereinto to provide space within which to retain further samples (e.g., from other target sites) without the need to remove the needle 102 from the body, as those skilled in the art will understand. In another embodiment of the invention, the stylet 108 may be completely withdrawn from the needle 102 after procuring a first sample. A new stylet (not shown) may then be inserted into the needle 102 while maintaining the position of the needle 102 in the body. Once the biopsy procedure has been completed (e.g., after multiple samples at different sites have been obtained), the stylet 108 is retracted to the closed configuration and the device 100 is withdrawn from the body. Subsequently, an endoscope or other visualization means may be inserted through the lumen 110 of the catheter shaft 106 to determine the efficacy of the procedure, as those skilled in the art will understand.

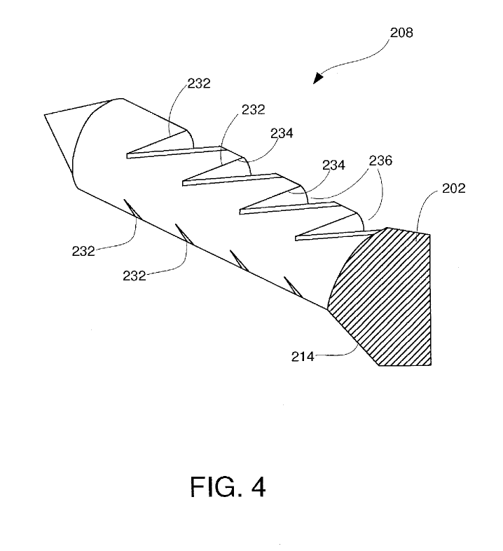

As shown in FIG. 4, a device according to an alternate embodiment of the invention includes a stylet 208 selectively movable within a needle 202 comprising a tapered portion 214 at a distal end thereof substantially similar to that described above in regard to the device 100. However, the stylet 208 comprises a series of sets of serrations 232, the sets being separated from one another longitudinally along the stylet 208. Each set of serrations 232 includes 2 serrations 232 with the serrations 232 of each set separated from one another about a circumference of the stylet 208 and facing away from a longitudinal axis of the stylet 208 on the same side of the axis. However, it is noted that any number of serrations may be included in each set and individual sets may differ from one another in the number of serrations 232 included therein to form any of a number of configurations without deviating from the scope of the present invention. The serrations 232 are formed as grooves tapering diagonally from enlarged ends at radially outer edges thereof to points within the body of the stylet 208. The grooves forming the serrations extend distally from the enlarged radially outer edges to form proximally leaning wedges. A wall 234 of each of the respective serrations 232 is sharpened to enable the anchoring and subsequent severing of tissue caught in a gap 236 between serrations 232 of adjacent sets, as described above with respect to the embodiment of FIGS. 1-3. The stylet 208 is also used in accordance with a method substantially similar to the stylet 108 of FIGS. 1-3.

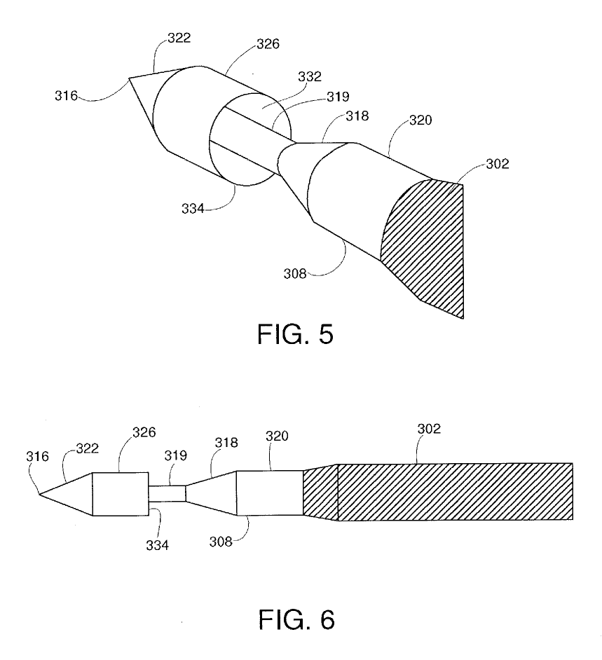

FIGS. 5-6 depict yet another alternate embodiment of the present invention, wherein a stylet 308 formed with a reverse funnel feature is shown in an open configuration. Specifically, the stylet 308 is formed as an elongated wire 320 formed with a substantially circular cross section. The elongated wire 320 further comprises a taper 318 tapering down to a reduced diameter wire 319. The reduced diameter wire 319 extends distally to a cup 326 having a diameter substantially equivalent to that of the elongated wire 320. The cup 326 further comprises a tapered distal tip 322 tapering down to a tissue puncturing tip 316. The cup 326 and the tapered distal tip 322 are both formed as hollow elements defining a cavity 332 therein serving to anchor the stylet 308 in place in the body by engaging tissue adjacent thereto when in the open configuration. If a tissue sampling procedure is to be performed, a sharpened rim 334 of the cup 326 serves to provide a cutting edge of the stylet 308 so that, upon actuation causing a proximal and distal movement thereof, the edge severs tissue received in the cavity 332 from surrounding tissue. It is noted that although the cup 326 is depicted with a 360° rim, the cup 326 may also comprise a less than a 360° rim depending on the requirements of the tissue being sampled.

In use, a needle 302 from which the stylet 308 projects distally is traversed to a target tissue sampling site with the stylet 308 drawn proximally into a lumen of the needle 302 until the tapered distal tip 322 projects from a tapered distal end of the needle 302 with the cup 326 received within the lumen of the needle 302. As in the previous embodiments, in this closed configuration, the tapered distal tip 322 may form a substantially continuous surface with the tapered distal end of the needle 302 to facilitate tissue penetration and minimize trauma. When the target site has been reached, the needle 302 is moved relative to the stylet 308 (e.g., by moving the stylet 308 distally while holding the needle 302 in position or by holding the stylet 308 in position while retracting the needle 302 proximally), as shown in FIGS. 5 and 6. As the stylet 308 is moved into the open configuration, surrounding tissue (e.g., target tissue from the target sampling site) is moved into the cavity 332 due to the pressure from the surrounding tissue anchoring the stylet 308 in place. The needle 302 may be then be moved proximally and distally until a desired sample of the target tissue has been captured in the cavity 332. Furthermore, the stylet 308 may also be moved proximally and distally to sever tissue within the cavity 332 from the surrounding tissue. As the stylet 308 is drawn into the needle lumen (not shown), suction may be applied thereto draw the tissue in the cavity 332 proximally and thus, permit multiple samplings by the stylet 308 without withdrawing the needle 302 from the body or by inserting a new stylet (not shown) into the needle 302 in the same manner described above.

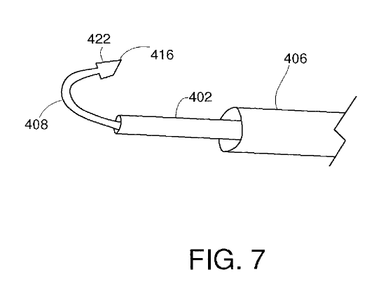

FIG. 7 depicts yet another embodiment of the present invention, in which a flexible stylet 408 is biased to assume a hooked shape when not constricted within a lumen of a needle 402. Specifically, the stylet 408 may be formed of a flexible material such as, for example, Nitinol. It is noted that although the present invention is described with the use of Nitinol, any other flexible material may he employed therein with the material being bent to a hook-like configuration. For example, the Nitinol of the stylet 408 may exhibit shape memory characteristics allowing the stylet 408 to revert to a desired memorized shape (e.g., the desired hook shape) when elevated to body temperature or may simply be biased toward this shape at all times when not constrained to a substantially straight shape within the lumen of the needle 402. In this manner, when the stylet 408 is inserted into a living body via a catheter 406 and moved distally out of the needle 402, the stylet 408 assumes the desired hooked configuration shown in FIG. 7 anchoring itself in surrounding tissue and resisting inadvertent removal of the stylet 408 and the needle 402 from the target position. Furthermore, a tapered tip 422 provided on the stylet 408 may be formed similar to the cup 326 of FIGS. 5-6. The tapered tip 422 may be made hollow to cut and capture tissue as it is drawn into engagement with the needle 402, as described in greater detail earlier. The tapered tip 422 further comprises a puncturing tip 416 which, when received at the distal end of the needle 402 facilitates insertion of the needle through intervening tissue in the same manner described above. It is further noted that the stylet 408 of the present invention may be modified in a number of ways without deviating from the scope of the invention to achieve desired anchoring and/or tissue capture properties. For example, the stylet 408 may be biased to assume a corkscrew shape at a distal end thereof to further anchor the needle 402 at the target site. Hoops of the corkscrew feature may be sharpened or otherwise treated to further aid in promoting severing of the sample tissue from surrounding tissue. The severed tissue may then be suctioned into the needle 402 in the same manner described above. It is further noted that although the present embodiment is depicted with one stylet 408 extending distally out of the needle 402, in another embodiment any plurality of hooked stylets may be employed wherein the plurality of hooks may all protrude distally out of the single needle 402 or from a respective number of separate needles 402 all stemming from the catheter 406.

The present invention may be applied to any procedure requiring the insertion of a needle into tissue via a device traversing a tortuous path. Though the present invention has been described with respect to the retrieval of tissue samples, it is submitted that devices for alternate uses such as, for example, the anchoring of needles for injection of fluids to or the withdrawal of fluids from the body may employ to invention without deviating from the spirit and scope of the present invention. Furthermore, although embodiments of the invention have been described with respect to a stylet movable with respect to a needle, alternate embodiments may also comprises a stylet and needle that are both movable relative to one another. In yet another embodiment, any portion of the needle or stylet may be provided with echosonic features to aid in location thereof when inserted to a target site in the body, as those skilled in the art will understand. Thus, these embodiments have been described in an exemplary manner and are not intended to limit the invention which is intended to cover all modifications and variations of this invention that come within the scope of the appended claims and their equivalents.

What is claimed is:

1. A device for capturing a tissue sample from within a body, comprising:
a needle comprising a needle lumen extending therethrough to a needle opening in the distal end; and
a stylet slidably received in the needle lumen for movement between an extended position in which a tissue penetrating distal tip of the stylet extends out of the needle opening to penetrate target tissue and a retracted position in which the distal tip of the stylet is received within the needle opening to substantially seal the needle lumen, the stylet further comprising an anchoring feature located proximally of the tissue penetrating distal tip, the anchoring feature comprising a first gripping member including a first proximal facing abutting surface adjacent to a first tissue receiving gap, movement to the extended position bringing the first gripping member into engagement with surrounding tissue to anchor the stylet at a desired position within the living body.

2. The device of claim 1, wherein the anchoring feature further comprises a second gripping member including a second proximal facing abutting surface, the second gripping member being separated from the first gripping member along a longitudinal axis of the stylet by the first tissue receiving gap.

3. The device of claim 2, wherein the first gripping member includes a plurality of first barbs separated from one another about a circumference of the stylet.

4. The device of claim 3, wherein first and second ones of the first barbs extend from the stylet on a same side of the longitudinal axis of the stylet.

5. The device of claim 3, wherein the plurality of first barbs includes 4 first barbs spaced substantially equally relative to one another about the longitudinal axis of the stylet.

6. The device of claim 3, wherein a radially outermost edge of each of the barbs is sharpened.

7. The device of claim 1, wherein the first gripping member comprises an edge formed as the first tissue receiving gap cut into the stylet.

8. The device of claim 7, wherein the edge is sharpened.

9. The device of claim 1, further comprising:
an actuator coupled to the proximal end of the needle, operation of the actuator moving the needle and the stylet proximally and distally relative to one another.

10. The device of claim 1, wherein the stylet is biased toward a hooked configuration so that the stylet bends away from a longitudinal axis of the needle when moved distally out of the needle lumen.

11. The device of claim 10, wherein the first gripping member is formed at a proximal end of an enlarged tissue penetrating tip of the stylet.

12. The device of claim 10, wherein the stylet is formed of a flexible material to assume a desired shape when extended from the needle lumen.

13. The device of claim 12, wherein the flexible material includes Nitinol.

14. The device of claim 10, wherein the enlarged tissue penetrating tip comprises an elongated cylindrical member extending proximally therefrom.

15. The device of claim 14, wherein the enlarged tissue penetrating tip of the stylet is hollow.

16. The device of claim 15, wherein the first tissue receiving gap is formed inside of the enlarged tissue penetrating tip of the stylet.

17. A device for capturing tissue samples from a body, comprising:
a needle comprising a needle lumen extending therethrough to a needle opening in the distal end;
a stylet slidably received in the needle lumen for movement between an extended position in which a tissue penetrating distal tip of the stylet extends out of the needle opening to penetrate target tissue and a retracted position in which the distal tip of the stylet is received within the needle opening to substantially seal the needle lumen, the stylet further comprising an anchoring feature located proximally of the tissue penetrating distal tip, movement to the extended position bringing the anchoring mechanism into engagement with surrounding tissue to anchor the stylet at a desired position within the living body; and
a tissue receiving space,
wherein the anchoring mechanism defines a shape and size of the tissue receiving space adjacent an outer wall of the stylet.

18. The device of claim 20, wherein the tissue receiving space is located distally of at least one tissue gripping member of the anchoring mechanism.

19. The device of claim 21, wherein tissue enters the tissue receiving space through an opening located adjacent the tissue gripping member.

20. A method for penetrating tissues within a living body, comprising:
inserting into a body to a location adjacent a target site a distal end of a needle, a needle lumen of the needle housing a stylet in a retracted position in which a distal tip of the stylet is received within a needle opening to substantially seal the needle lumen;
exposing an anchoring feature located along a distal length of the stylet, the anchoring feature comprising a first gripping member including a first proximal facing abutting surface adjacent to a first tissue receiving gap, to bring the first gripping member into engagement with surrounding tissue;
anchoring the stylet at a desired position within the living body;
moving the needle relative to the stylet to capture a tissue sample in the needle lumen; and
moving the stylet back into closed engagement with the needle opening with the anchoring mechanism housed within the needle lumen.

21. The method of claim 20, wherein the stylet is exposed by retracting the needle proximally while maintaining a position of the stylet substantially constant.

22. The method of claim 20, wherein the stylet is exposed by advancing the stylet distally from the needle opening while maintaining a position of the needle substantially constant.
